(19) United States
(12) Patent Application Publication
(10) Pub. No.: US 2025/0025633 A1
(43) Pub. Date: Jan. 23, 2025

(54) Sub-assembly for a medicament delivery device

(71) Applicant: SHL Medical AG, Zug (CH)

(72) Inventors: Daniel Carlsson, Nacka Strand (SE); Oscar Alexandersson, Nacka Strand (SE); Johan Zander, Nacka Strand (SE)

(21) Appl. No.: 18/706,823

(22) PCT Filed: Oct. 24, 2022
(86) PCT No.: PCT/EP2022/079661

§ 371 Date: May 2, 2024

(30) Foreign Application Priority Data
Nov. 5, 2021 (EP) 21206708.6

(51) Int. Cl. A61M 5/315

(52) U.S. Cl. CPC A61M 5/315

(57) ABSTRACT

The present disclosure provides a sub-assembly for a medicament delivery device. The sub-assembly includes a housing and a delivery member cover arranged in the housing and configured to move axially in the housing from an extended position towards a retracted position. The delivery member cover includes a radial wall provided with an axial through-opening. The sub-assembly further includes a removable cap assembly. The removable cap assembly includes a cap configured to be fitted to the delivery member cover and/or to the housing, a distal cap end, and a delivery member shield gripping sleeve having a radially flexible axially extending arm.

### CROSS-REFERENCE TO RELATED APPLICATIONS

The present application is a U.S. National Phase Application pursuant to 35 U.S.C. § 371 of International Application No. PCT/EP2022/079661 filed Oct. 24, 2022, which claims priority to European Patent Application No. 21206708.6 filed Nov. 5, 2021. The entire disclosure contents of these applications are herewith incorporated by reference into the present application.

### TECHNICAL FIELD

The present disclosure generally relates to medicament delivery devices.

BACKGROUND

Medicament delivery devices such as autoinjectors may comprise a housing and a needle cover that is movable relative to the housing. The needle cover may protrude from the housing in an initial state of the medicament delivery device. By pushing the needle cover further into the housing the device may be activated. This is typically performed by pressing the medicament delivery device towards the injection site. Due to this construction, there is a risk of unintended activation of the medicament delivery device for example by accidentally dropping the device.

### SUMMARY

An object of the present disclosure is to provide a sub-assembly for a medicament delivery device which solves, or at least mitigates, problems of the prior art.

There is hence according to a first aspect of the present disclosure provided a sub-assembly for a medicament delivery device, comprising: a housing, a delivery member cover arranged in the housing, and configured to move axially in the housing from an extended position towards a retracted position, the delivery member cover having a radial wall provided with an axial through-opening, and a removable cap assembly comprising: a cap configured to be fitted to the delivery member cover and/or to the housing, the cap having a proximal cap end and a distal cap end, a delivery member shield gripping sleeve having a radially flexible axially extending arm, wherein the delivery member shield gripping sleeve is arranged in the cap and the delivery member shield gripping sleeve is configured to slide in the cap towards the distal cap end from an initial position in which the arm extends through the through-opening of the delivery member cover, to a second position when the cap is pulled from the delivery member cover, wherein the cap assembly comprises an axial structure configured to extend parallel with the delivery member shield gripping sleeve when the delivery member shield gripping sleeve is in the initial position to restrict radial inwards movement of the arm so that the arm has a radial dimension that extends beyond a radial dimension of the through-opening, thereby preventing the delivery member cover to move towards the retracted position, wherein the delivery member shield gripping sleeve is configured to move relative to the axial structure when the delivery member shield gripping sleeve is moved towards the second position, enabling the arm to flex radially inwards and move through the through-opening of the delivery member cover.

The radially flexible axially extending arm thus restricts movement of the delivery member cover towards its retracted position in case a medicament delivery device containing the sub-assembly is subjected to a shock for example due to being accidentally dropped. Thus, in case the delivery member cover is configured to trigger medicament delivery by moving to the retracted position, accidental triggering may be avoided.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the cap comprises a proximally directed radial stop surface and the delivery member shield gripping sleeve comprises a distally directed sleeve radial surface facing and being axially spaced apart from the radial stop surface when the delivery member shield gripping sleeve is in the initial position, the sleeve radial surface being configured to contact the radial stop surface when the delivery member shield gripping sleeve is in the second position.

The delivery member shield gripping sleeve is thus prevented from moving further than to the second position when moving from the initial position.

According to one embodiment the delivery member shield gripping sleeve has an axial slot that is provided with the sleeve radial surface, and the radial stop surface is provided on a radially inwards extending protrusion extending into the slot with a play to delimit axial movement of the delivery member shield gripping sleeve between the initial position and the second position.

According to one embodiment the arm and the axial structure form a torsional snap fit with the radial wall.

According to one embodiment the arm is a first arm extending towards the distal cap end and forms part of a see-saw structure comprising a radially flexible second arm extending axially towards the proximal cap end, wherein the axial structure is arranged radially outwards of the second arm urging the second arm radially inwards when the delivery member shield gripping sleeve is in the initial position, causing the first arm to flex radially outwards and obtain a radial dimension that is greater than a radial dimension of the through-opening.

According to one embodiment the axial structure has an inclined inner surface configured to bear against and urge the second arm radially inwards, the inclined surface increasing a radial dimension of the axial structure in a direction towards the proximal cap end.

According to one embodiment the delivery member shield gripping sleeve has a radially outwards extending sleeve flange defining the sleeve radial surface, and the radial stop surface extends radially inwards and is arranged axially spaced apart from the sleeve radial surface when the delivery member shield gripping sleeve is in the initial position, the radial stop surface being configured to bear against the sleeve flange when the delivery member shield gripping sleeve is in the second position.

According to one embodiment the axial structure is arranged radially inwards of the arm.

According to one embodiment the arm includes an inclined portion that gradually increases a radial dimension of the delivery member shield gripping sleeve in a direction towards a distal sleeve end of the delivery member shield gripping sleeve when the delivery member shield gripping sleeve is in the initial position, the inclined portion having a maximum radial dimension that is greater than the radial dimension of the through-opening.

According to one embodiment the radial wall has a distally directed wall surface, wherein the inclined portion is arranged distally relative to the distally directed wall surface when the delivery member shield gripping sleeve is in the initial position.

According to one embodiment the delivery member shield gripping sleeve has radially inwards extending flexible tabs configure to engage with a delivery member shield.

According to one embodiment the delivery member shield gripping sleeve extends out from the cap at the distal cap end, wherein the arm is arranged at a proximal portion of the delivery member shield gripping sleeve, and the tabs are arranged at a distal portion of the delivery member shield gripping sleeve.

According to one embodiment the housing has a proximally facing surface configured to bear against the cap when the cap is fitted to the delivery member cover.

There is according to a second aspect provided a medicament delivery device comprising the sub-assembly of the first aspect.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an example of a medicament delivery device;

FIG. 2 shows an exploded view of the medicament delivery device in FIG. 1;

FIG. 3 is a perspective view of a delivery member cover;

FIG. 4 shows an example of a cap assembly;

FIG. 5 depicts a longitudinal section of the cap of the cap assembly in FIG. 4;

FIG. 6 shows a longitudinal section of a proximal portion of the medicament delivery device in FIG. 1 before the cap assembly in FIG. 4 is removed;

FIG. 7 is a longitudinal section of a proximal portion of the medicament delivery device in FIG. 1 when the cap assembly is being removed;

FIG. 8 is another example of a cap assembly;

FIG. 9 shows a perspective view of a cap of the cap assembly in FIG. 8;

FIG. 10 shows a longitudinal section of a proximal portion of a medicament delivery device before the cap assembly in FIG. 8 is removed; and

FIG. 11 depicts a longitudinal section of a proximal portion of a medicament delivery device when the cap assembly in FIG. 8 is being removed.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

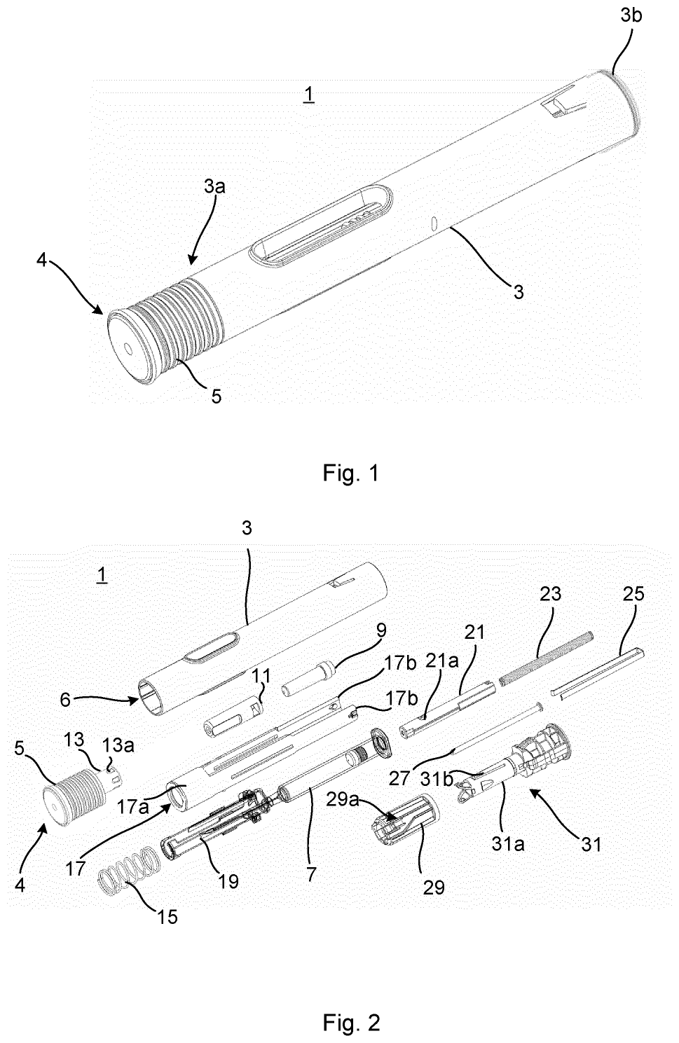

With reference to FIGS. 1-11 a sub-assembly for a medicament delivery device will herein be described. The sub-assembly comprises a housing 3, a delivery member cover 17 protruding from a proximal end opening of the housing 3 and a removable cap assembly 4, 4′.

The sub-assembly is configured to prevent the medicament delivery device from unintentionally triggering. The medicament delivery device comprising the assembly is designed to be triggered by an axial movement of the medicament delivery member cover relative to the housing. This axial movement of the medicament delivery cover relative to the housing results in that a medicament contained in the medicament delivery device is delivered to a medicament delivery site.

A detailed explanation of the assembly is provided below in the context of one main example of the medicament delivery device. It should be noted that the assembly may also be used with other types of medicament delivery devices that are triggered by axial movement of the delivery member cover relative to the housing.

FIG. 1 shows an example of a medicament delivery device 1 that comprises the above-mentioned sub-assembly. The medicament delivery device 1 is in the present example an autoinjector.

The medicament delivery device 1 comprises the housing 3. The housing 3 of the sub-assembly has a proximal end 3a and a distal end 3b. The housing 3 is hollow and may be generally tubular. The housing 3 may comprise a proximal end opening.

The medicament delivery device 1 comprises the cap assembly 4. The cap assembly 4 of the sub-assembly comprises a cap 5. The cap 5 is configured to be fitted to the proximal end 3a of the housing 3 and/or onto a delivery member cover protruding from the proximal end opening of the housing 3.

FIG. 2 is an exploded view of the medicament delivery device 1.

The medicament delivery device 1 is configured to accommodate a medicament container such as a syringe 7. The syringe 7 comprises a delivery member, in this case a needle. A delivery member shield may be provided around the delivery member. The delivery member shield may for example comprise a flexible needle shield 9 and/or a rigid needle shield 11. In case it comprises the flexible needle shield 9 and the rigid needle shield 11, the rigid needle shield 11 is arranged radially outside the flexible needle shield 9 may be mounted to the delivery member.

The cap assembly 4 of the sub-assembly comprises a delivery member shield gripping sleeve 13 configured to engage with the rigid needle shield 11. The delivery member shield gripping sleeve 13 may for example comprise radially inwards extending tabs 13a configured to engage with the rigid needle shield 11. When the cap 5 is removed, it brings with it the flexible needle shield 9 and the rigid needle shield 11.

The medicament delivery device comprises the aforementioned delivery member cover 17. The delivery member cover 17 is slidably arranged in the housing 3 and extends from the housing 3 from the proximal end opening 6 of the housing 3.

The delivery member cover 17 is configured to move axially relative to the housing 3 from an extended position to a retracted position in which the housing 3 receives a larger portion of the delivery member cover 17 than in the extended position.

The delivery member cover 17 is biased towards the extended position. The medicament delivery device 1 comprises a first resilient member 15 configured to bias the delivery member cover 17 towards the extended position. The first resilient member 15 may for example be a coil spring.

The first resilient member 15 may for example be arranged to extend inside the delivery member cover 17 between a radial surface of the delivery member cover 17 and a radial surface provided inside the housing 3.

The delivery member cover 17 has a proximal tubular portion 17a and legs 17b extending distally towards the distal end 3b of the housing 3.

The medicament delivery device 1 comprises a medicament container holder 19. The medicament container holder 19 is arranged in the housing 3 and is configured to hold a medicament container such as the syringe 7.

The housing 3 may for example comprise a radially inwards extending ledge which prevents the medicament container holder 19 from moving in the proximal direction, i.e., towards the proximal end 3a of the housing 3.

The medicament delivery device 1 comprises a plunger rod 21. The plunger rod 21 is configured to extend into the medicament container, i.e., the syringe 7 in this example.

The plunger rod 21 is configured to be moved in the proximal direction inside the housing 3. The plunger rod 21 is configured to be moved from an initial axial position to a final position, which is closer to the proximal end 3a of the housing 3 than the initial axial position.

In a preferred example, the plunger rod 21 comprises radial recesses 21a. Alternatively, the plunger rod comprises a ledge extending from an outer surface of a wall of the plunger rod in the radial direction. In a preferred example, the plunger rod 21 is hollow. Alternatively, the plunger rod comprises a tubular section and a rod section.

In a preferred example, the medicament delivery device 1 comprises a rod 23 and a second resilient member 25. The rod 23 is configured to be arranged in the plunger rod 21 and the second resilient member 25, for example a spring, may be arranged around the rod 23 in the plunger rod 21. Alternatively, the second resilient member 25 may be arranged partially around the plunger rod, in which case the medicament delivery device is without the rod 23.

In another example, the medicament delivery device comprises a gas canister and a valve connected to the gas canister for driving the plunger rod from the initial axial position.

In one example, where the medicament delivery device comprises the rod 23 and the second resilient member 25, the medicament delivery device 1 may comprise a U-shaped bracket 27. The U-shaped bracket 27 is arranged with its bottom of the U-shape facing towards the distal end 3b of the housing 3. The second resilient member 25 is configured to bias the U-shaped bracket 27 towards the distal end 3b of the housing 3.

In a preferred example, the medicament delivery device 1 comprises a rotator 29 and a rear cap structure 31.

In one example where the plunger rod 21 is biased in the proximal direction relative to the housing 3 by the second resilient member 25, the rotator 29 and the rear cap structure 31 are configured to releasably hold the plunger rod 21 against a biasing force from the second resilient member 25. The rear cap structure 31 is configured to engage with the plunger rod 21 to maintain the plunger rod 21 in its initial axial position. The rear cap structure 31 has a tubular proximal portion 31a provided with radially flexible arms 31b. Each flexible arm 31b is configured to engage with a respective radial recess 21a of the plunger rod 21 before the medicament delivery device 1 has been activated or triggered to perform medicament delivery.

The rotator 29 is arranged radially outside of and around the tubular proximal portion 31a.

The rotator 29 is configured to rotate relative to the tubular proximal portion 31a from a first rotational position, which it holds prior to activation of the medicament delivery device 1, to a second rotational position.

The rotator 29 has an inner surface which when the rotator 29 is in the first rotational position bears against the flexile arms 31b, preventing the flexible arms 31b to disengage from a respective one of the radial recesses 21a.

The rotator 29 has an inner surface provided with inner recesses or windows which are arranged circumferentially offset from the flexible arms 31b when the rotator 29 is in the first rotational position.

The rotator 29 is configured to cooperate with the delivery member cover 17 when the delivery member cover 17 is moved from the extended position towards the retracted position. The rotator 29 is configured to translate linear movement of the delivery member cover 17 to rotation. The rotator 29 has a guide structure 29a configured to cooperate with radially inwards extending pins of the legs 17b of the delivery member cover 17 such that when the delivery member cover 17 is moved towards the retracted position the pins move against cam surfaces of the guide structure 29a. This causes rotation of the rotator 29 from the first rotational position to the second rotational position. When the delivery member cover 17 reaches the retracted position, the inner recesses, or windows, of the inner surface of the rotator 29 align with the flexible arms 31b of the rear cap structure 31. The flexible arms 31b are thereby able to flex radially outwards and disengage from the radial recesses 21a of the plunger rod 21 and the plunger rod 21 is thus released to move axially from the initial axial position towards the final position.

When the plunger rod 21 is released and reaches its final position, the U-shaped bracket 25 which previously was in engagement with the rear cap structure 31 is released and impacts with a radial surface, e.g., inside the rear cap structure 31. This causes an audible click, indicating that the medicament delivery operation has been finalised.

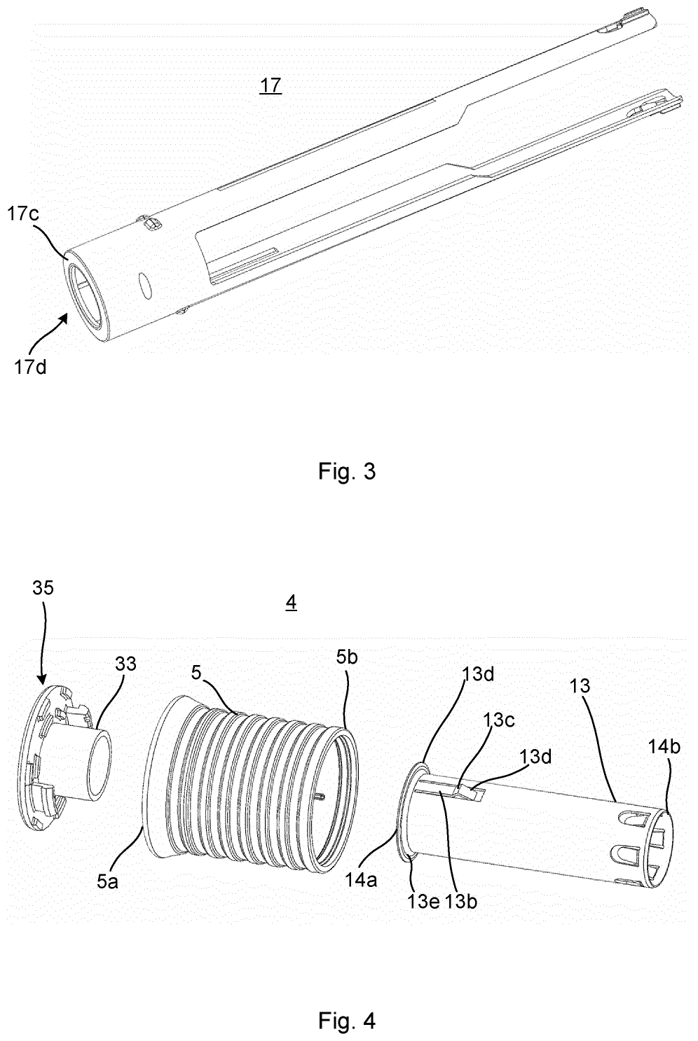

FIG. 3 shows a perspective view of the delivery member cover 17. The delivery member 17 comprises a radial wall 17c. The radial wall 17c forms a proximal end of the delivery member cover 17. The radial wall 17c forms a proximal end of the proximal tubular portion 17a.

The radial wall 17c is provided with an axial through-opening 17d. The through-opening 17d leads into an interior of the proximal tubular portion 17a.

FIG. 4 shows the cap assembly 4. The exemplified cap assembly 4 comprises a front part 35 configured to be attached to the cap 5. The front part 35 forms a proximal end of the cap assembly 4.

The cap 5 has a proximal cap end 5a and a distal cap end 5b. The front part 35 is configured to be connected to the cap 5 at the proximal cap end 5a.

The cap 5 is hollow and open at the distal cap end 5b. The cap 5 is configured to receive a proximal portion of the delivery member shield gripping sleeve 13.

The delivery member shield gripping sleeve 13 extends in the distal direction beyond the distal cap end 5b of the cap 5. The delivery member shield gripping sleeve 13 protrudes from the distal cap end 5b of the cap 5.

The delivery member shield gripping sleeve 13 has a proximal sleeve end 14a and a distal sleeve end 14b.

The delivery member shield gripping sleeve 13 has a radially flexible axially extending arm 13b. The arm 13b extends in a distal direction, in a direction from the proximal sleeve end 14a towards the distal sleeve end 14b.

The arm 13b extends distally from a proximal end portion of the delivery member shield gripping sleeve 13. The tabs 13a are provided at a distal end portion of the delivery member shield gripping sleeve 13.

The through-opening 17d is configured to receive a portion of the delivery member shield gripping sleeve 13, including at least a portion of the arm 13b.

The arm 13b has an inclined portion 13c which gradually increases a radial dimension of the delivery member shield gripping sleeve 13 in a direction towards the distal end of the delivery member shield gripping sleeve 13. The inclined portion 13c has a maximum radial dimension that is greater than the radial dimension of the through-opening 17d.

The arm 13b also has an inclined portion 13d directly following the inclined portion 13c. The inclined portion 13d gradually decreases a radial dimension of the delivery member shield gripping sleeve 13 in a direction towards the distal end of the delivery member shield gripping sleeve 13. This facilitates insertion of the delivery member shield gripping sleeve 13 into the through-opening 17d during assembly of the medicament delivery device 1.

The delivery member shield gripping sleeve 13 has a radially outwards extending sleeve flange 13d. The sleeve flange 13d forms the proximal sleeve end 14a.

The sleeve flange 13d defines a sleeve radial surface 13e. The sleeve radial surface 13e is directed distally. The sleeve radial surface 13e thus faces the distal end 3b of the housing 3 when the cap assembly 4 has been fitted to the delivery member cover 17.

The delivery member shield gripping sleeve 13 is slidably arranged in the cap 5. The delivery member shield gripping sleeve 13 is configured to be moved inside the cap 5 from an initial position to a second position relative to the cap 5.

The cap assembly 4 comprises an axial structure 33. The axial structure 33 extends axially inside the cap 5. The axial structure 33 may for example be tubular or cylindrical. The axial structure 33 extends axially towards the distal cap end 5b of the cap 5.

The front part 35 is in the present example provided with the axial structure 33.

The axial structure 33 is configured to be arranged adjacent to and radially inwards of the arm 13b when the delivery member shield gripping sleeve 13 is in the initial position. For example, when the axial structure 33 is tubular, the axial structure 33 is configured to be arranged concentrically with and radially inwards of the arm 13b when the delivery member shield gripping sleeve 13 is in the initial position. Alternatively, the axial structure can be a wall extending parallel with and radially inwards of the arm, adjacent to the arm when the delivery member shield gripping sleeve is in the initial position. The axial structure 33 is radially arranged so close to the arm 13b that the arm 13b is restricted to flex radially inwards. The axial structure 33 is configured to restrict the inclined portion 13c to flex radially inwards which such an amount that it can pass through the through-opening 17d after the cap assembly 4 has been fitted to the delivery member cover 17 when the delivery member shield gripping sleeve 13 is in the initial position.

In one example of assembling the sub-assembly, when the cap assembly 4 is fitted to the delivery member cover 17 in the assembly process, the cap 5 containing the delivery member shield gripping sleeve 13 is slid over the delivery member cover 17 from a proximal end of the delivery member cover 17. At this point, the front part 35 has generally not yet been fitted, allowing the arm 13b to flex radially inwards as the delivery member shield gripping sleeve 13 moves through the through-opening 17d. The front part 35 is then attached to the cap 5. The axial structure 33 is thus moved inside the delivery member shield gripping sleeve 13 radially inwards of the arm 13b. This restricts radial inwards movement of the arm 13b.

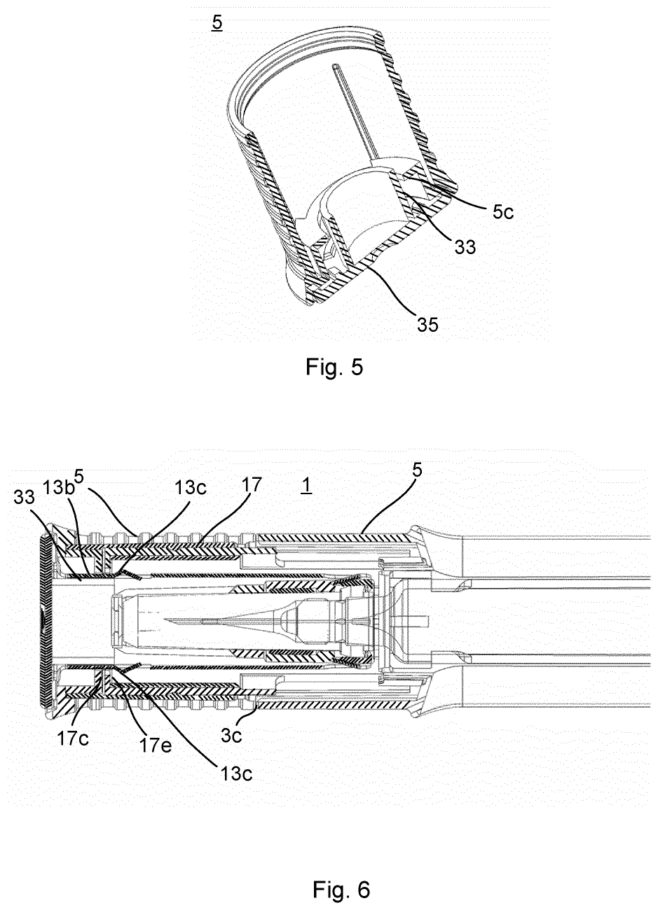

FIG. 5 shows a longitudinal section of the cap 5. The cap 5 has a proximally directed radial stop surface 5c. The radial stop surface 5c is configured to face the sleeve radial surface 13e. When the delivery member shield gripping sleeve 13 is in the initial position, the sleeve radial surface 13e is axially spaced apart from the radial stop surface 5c. When the cap assembly 4 is pulled from the delivery member cover 17, the delivery member shield gripping sleeve 13 maintains its axial position relative to the delivery member cover 17 until the radial stop surface 5c has reached the sleeve radial surface 13e. The delivery member shield gripping sleeve 13 thus reaches its second position.

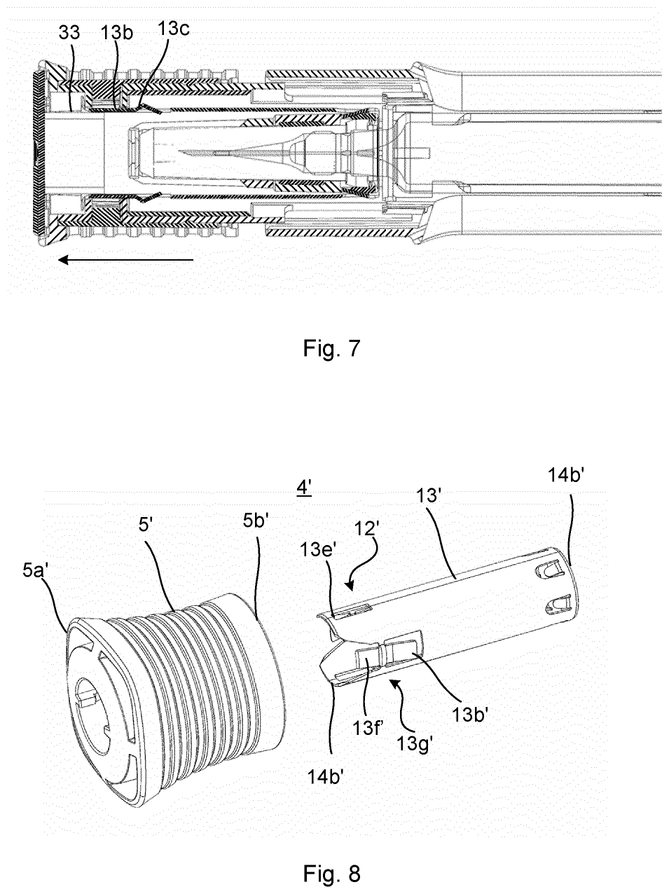

Removal of the cap assembly 4 from the delivery member cover 17 will now be described with reference to FIGS. 6-7.

In FIG. 6, the cap assembly 4 is fitted to the delivery member cover 17.

The housing 3 has a proximally facing surface 3c which bears against the distal cap end 5b.

The delivery member shield gripping sleeve 13 is arranged in the initial position relative to the cap 5. The axial structure 33 is arranged radially inwards of the arm 13b and extends axially up to or essentially up to the inclined portion 13c of the arm 13b. The arm 13b is thus restricted to flex radially inwards. The arm 13b extends through the through-opening 17d of the delivery member cover 17.

The radial wall 17c of the delivery member cover 17 has a distally directed wall surface 17e. The distally directed wall surface 17e is arranged proximally relative to the inclined portion 13c. The inclined portion 13c is arranged close enough to the distally directed wall surface 17e that impact or shock on the medicament delivery device 1 will not lead to an axial movement of the delivery member cover 17 to such a degree as to cause activation of the medicament delivery device 1. In this example, this means that the rotator 29 should not be able to move to the second rotational position. The inclined portion 13c may for example be arranged adjacent to or bear against the distally directed wall surface 17e when the delivery member shield gripping sleeve 13 is in the initial position.

The distal sleeve end 14b of the delivery member shield gripping sleeve 13 is arranged facing and adjacent to a proximally directed surface of the housing 3 inside the housing 3 when the cap 5 has been fitted to the delivery member cover 17. Thus, for example, in case the medicament delivery device 1 is subjected to an impact, e.g., due to being dropped, the delivery member shield gripping sleeve 13 will hit the proximally directed surface of the housing 3 thus restricting its movement in the distal direction, i.e., towards the distal end 3b of the housing 3. FIG. 7 illustrates when the cap assembly 4 is in the midst of being pulled off the delivery member cover 17. Since the delivery member shield gripping sleeve 13 is slidably arranged in the cap 5, the delivery member shield gripping sleeve 13 initially maintains its axial position relative to the delivery member cover 17 when the cap 5 is pulled off. When the cap 5 has moved such a distance that the radial stop surface 5c contacts the sleeve radial surface 13e, as shown in FIG. 7, the delivery member shield gripping sleeve 13 is finally brought off with the cap 5. At this point, most of the axial structure 33 has been moved out from underneath the arm 13b. This enables the arm 13b to flex radially inwards as the cap 5 is continued to be pulled from the delivery member cover 17, and the delivery member shield gripping sleeve 13 is moved concurrently with the cap 5. The arm 13b may thus flex radially inwards as the inclined portion 13c is pulled against the distally directed wall surface 17e. The arm 13b will thus be able to move through the through-opening 17d of the delivery member cover 17. The delivery member shield gripping sleeve 13 may thus be pulled out from the delivery member cover 17 and the cap assembly 4 can be separated from the housing 3 and the delivery member cover 17.

FIG. 8 shows another example of a cap assembly. The cap assembly 4′ comprises a cap 5′ and a delivery member shield gripping sleeve 13′.

The delivery member shield gripping sleeve 13′ has an axial slot 12′. The axial slot 13e′ is provided on a proximal portion of the delivery member shield gripping sleeve 13′.

The axial slot 12′ has a proximal slot end and a distal slot end. The proximal slot end has a distally directed sleeve radial surface 13e′.

The delivery member shield gripping sleeve 13′ has radially flexible axially extending arm 13b′. The arm 13b′ extends in a distal direction, in a direction from a proximal sleeve end 14a′ towards a distal sleeve end 14b′.

The arm 13b′ extends distally from a proximal end portion of the delivery member shield gripping sleeve 13′.

The arm 13b′ is a first arm extending distally and forms part of a see-saw structure 13g′ comprising a radially flexible second arm 13f extending axially in the proximal direction. The arm 13b′ and the second arm 13f thus extend in opposite axial directions.

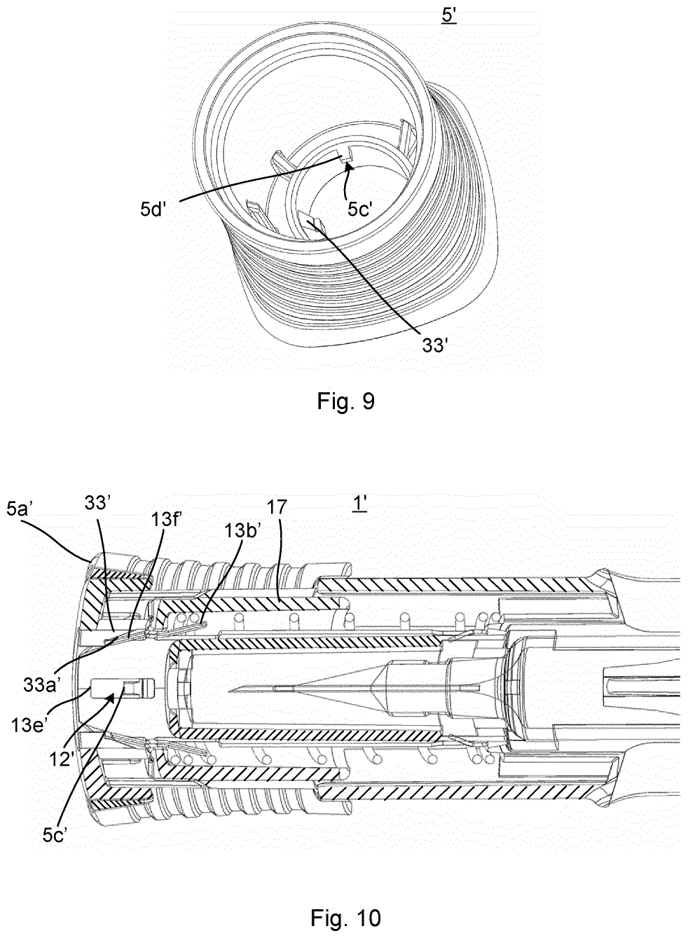

FIG. 9 shows a perspective view of the interior of the cap 5′. The cap 5′ has a proximally directed radial stop surface 5c′. The radial stop surface 5c′ is provided on a radially inwards extending protrusion 5d′ of the cap 5′. The protrusion 5d′ extends into the slot 12′. There is a play between the protrusion 5d′ and the slot 12′ so that the protrusion 5d′ can move in the slot 12′.

Like in the previous example, the delivery member shield gripping sleeve 13′ is configured to slide inside the cap 5′, from an initial position to a second position relative to the cap 5′. When the delivery member shield gripping sleeve 13′ is in the initial position, the protrusion 5d′ and the radial stop surface 5c′ are arranged distanced from the sleeve radial surface 13e′. The delivery member shield gripping sleeve 13′ obtains the second position when the cap 5′ is pulled from the delivery member cover 17 and the radial stop surface 5c′ reaches the sleeve radial surface 13e′. The slot 12′ thus delimits axial movement of the delivery member shield gripping sleeve 13′ relative to the cap 5′.

The cap 5′ is in this example provided with the axial structure 33′. The axial structure 33′ extends axially inside the cap 5′. The axial structure 33′ is arranged offset from the protrusion 5d′ in the circumferential direction of the cap 5′. This offset may for example be 90 degrees.

The axial structure 33′ is arranged radially outside of and bears against the second arm 13f when the delivery member shield gripping sleeve 13′ is in the initial position, as shown in FIG. 10.

Turning to FIG. 10, the cap assembly 4′ is shown fitted to the delivery member cover 17 before removal.

The arm 13b′ is received by the through-opening 17d of the delivery member cover 17 when the delivery member shield gripping sleeve 13′ is in the initial position and the cap 5′ is fitted to the delivery member cover 17.

The axial structure 33′ has an inclined surface 33a′ by means which a radial dimension, or radial thickness, of the axial structure 33′ increases in a direction towards the proximal cap end 5a′. The inclined surface 33a′ urges the second arm 13f′ radially inwards. Due to the see-saw structure 13g′, the arm 13b′ is flexed radially outwards. The arm 13b′ thus has an inclined portion which increases the radial dimension of the arm 13b′ gradually in a direction towards the distal sleeve end 14b′. The arm 13b′ obtains a radial dimension which is greater than a corresponding radial dimension of the through-opening 17d. The delivery member cover 17 is therefore prevented from moving to the retracted position, for example in case the medicament delivery device 1′ fitted with the cap assembly 4′ is subjected to an impact or shock.

Like in the previous example, the delivery member shield gripping sleeve 13′ is restricted to move in the distal direction to due to the distal sleeve end 14b′ facing and being arranged adjacent to a proximally directed surface of the housing 3 inside the housing 3.

In FIG. 10, the sleeve radial surface 13e′ is axially spaced apart from the radial stop surface 5c′.

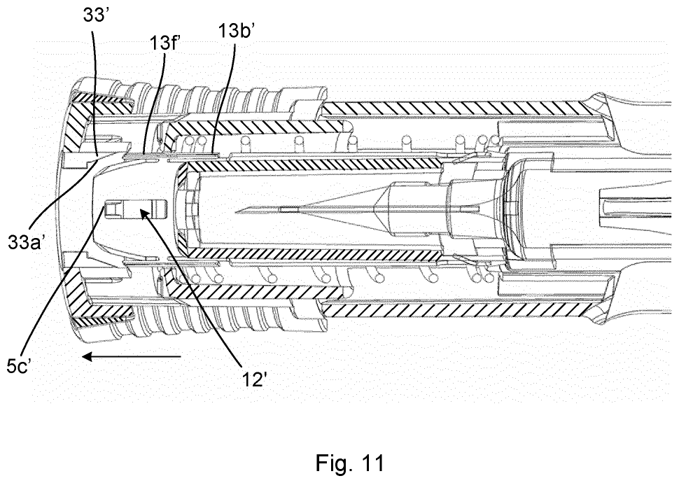

FIG. 11 shows when the cap assembly 4′ is in the process of being pulled of the delivery member cover 17. The radial stop surface 5c′ has reached the sleeve radial surface 13e′ and the delivery member shield gripping sleeve 13′ has thus moved to the second position in the cap 5′.

The axial structure 33′ has moved in the proximal direction away from the second arm 13f which thus flexes out radially. This causes the arm 13b′ to flex radially inwards. The arm 13b′ is thus able to pass through the through-opening 17d.

Since the radial stop surface 5c′ engages with the sleeve radial surface 13e′, the cap 5 and the delivery member shield gripping sleeve 13′ can now be pulled off the delivery member cover 17.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. A sub-assembly for a medicament delivery device, comprising:
a housing;
a delivery member cover arranged in the housing and configured to move axially in the housing from an extended position towards a retracted position, the delivery member cover having a radial wall provided with an axial through-opening; and
a removable cap assembly, the removable cap assembly comprising:
a cap configured to be fitted to the delivery member cover and/or to the housing, the cap having a proximal cap end and a distal cap end;
a delivery member shield gripping sleeve having a radially flexible axially extending arm,
wherein the delivery member shield gripping sleeve is arranged in the cap and the delivery member shield gripping sleeve is configured to slide in the cap towards the distal cap end from an initial position in which the arm extends through the through-opening of the delivery member cover, to a second position when the cap is pulled from the delivery member cover,
wherein the cap assembly comprises an axial structure configured to extend parallel with the delivery member shield gripping sleeve when the delivery member shield gripping sleeve is in the initial position to restrict radial inwards movement of the arm so that the arm has a radial dimension that extends beyond a radial dimension of the through-opening, thereby preventing the delivery member cover to move towards the retracted position, and
wherein the delivery member shield gripping sleeve is configured to move relative to the axial structure when the delivery member shield gripping sleeve is moved towards the second position, enabling the arm to flex radially inwards and move through the through-opening of the delivery member cover.

16. The sub-assembly as claimed in claim 15, wherein the cap comprises a proximally directed radial stop surface and the delivery member shield gripping sleeve comprises a distally directed sleeve radial surface facing and being axially spaced apart from the radial stop surface when the delivery member shield gripping sleeve is in the initial position, the sleeve radial surface being configured to contact the radial stop surface when the delivery member shield gripping sleeve is in the second position.

17. The sub-assembly as claimed in claim 16, wherein the delivery member shield gripping sleeve has an axial slot that is provided with the sleeve radial surface, and the radial stop surface is provided on a radially inwards extending protrusion extending into the slot with a play to delimit axial movement of the delivery member shield gripping sleeve between the initial position and the second position.

18. The sub-assembly as claimed in claim 16, wherein the arm and the axial structure form a torsional snap fit with the radial wall.

19. The sub-assembly as claimed in claim 17, wherein the arm is a first arm extending towards the distal cap end and forms part of a see-saw structure comprising a radially flexible second arm extending axially towards the proximal cap end, wherein the axial structure is arranged radially outwards of the second arm urging the second arm radially inwards when the delivery member shield gripping sleeve is in the initial position, causing the first arm to flex radially outwards and obtain a radial dimension that is greater than a radial dimension of the through-opening.

20. The sub-assembly as claimed claim 19, wherein the axial structure has an inclined inner surface configured to bear against and urge the second arm radially inwards, the inclined surface increasing a radial dimension of the axial structure in a direction towards the proximal cap end.

21. The sub-assembly as claimed in claim 16, wherein the delivery member shield gripping sleeve has a radially outwards extending sleeve flange defining the sleeve radial surface, and the radial stop surface extends radially inwards and is arranged axially spaced apart from the sleeve radial surface when the delivery member shield gripping sleeve is in the initial position, the radial stop surface being configured to bear against the sleeve flange when the delivery member shield gripping sleeve is in the second position.

22. The sub-assembly as claimed in claim 21, wherein the axial structure is arranged radially inwards of the arm.

23. The sub-assembly as claimed in claim 15, wherein the arm includes an inclined portion that gradually increases a radial dimension of the delivery member shield gripping sleeve in a direction towards a distal sleeve end of the delivery member shield gripping sleeve when the delivery member shield gripping sleeve is in the initial position, the inclined portion having a maximum radial dimension that is greater than the radial dimension of the through-opening.

24. The sub-assembly as claimed in claim 23, wherein the radial wall has a distally directed wall surface, wherein inclined portion is arranged distally relative to the distally directed wall surface when the delivery member shield gripping sleeve is in the initial position.

25. The sub-assembly as claimed in claim 15, wherein the delivery member shield gripping sleeve has radially inwards extending flexible tabs configure to engage with a delivery member shield.

26. The sub-assembly as claimed in claim 25, wherein the delivery member shield gripping sleeve extends out from the cap at the distal cap end, wherein the arm is arranged at a proximal portion of the delivery member shield gripping sleeve, and the tabs are arranged at a distal portion of the delivery member shield gripping sleeve.

27. The sub-assembly as claimed in claim 15, wherein the housing has a proximally facing surface configured to bear against the cap when the cap is fitted to the delivery member cover.

28. A medicament delivery device comprising the sub-assembly as claimed in claim 15.
